(19) United States
(12) Patent Application Publication
(10) Pub. No.: US 2022/0133672 A1
(43) Pub. Date: May 5, 2022

(54) NOTCH INHIBITORS FOR THE TREATMENT OF VASCULAR MALFORMATIONS

(71) Applicant: The University of North Carolina at Chapel Hill, Chapel Hill, NC (US)

(72) Inventors: Julie M. Blatt, Chapel HI!!, NC (US); Reema B. Davis, Apex, NC (US); Kristy Pahl, Chapel Hill, NC (US); Kathleen Marie-lsabelle Caron, Chapel Hill, NC (US); Scott Victor Smith, Cary, NC (US); Carrie J. Shawber, Township of Washington, NC (US)

(21) Appl. No.: 17/415,813

(22) PCT Filed: Dec. 18, 2019
(86) PCT No.: PCT/US19/67062

§ 371 Date: Jun. 18, 2021

Related U.S. Application Data
(60) Provisional application No. 62/781,473, filed on Dec. 18, 2018.

(51) Int. Cl. A61K 31/216; A61P 9/00; A61K 31/55

(52) U.S. Cl. CPC A61K 31/216; A61K 31/55; A61P 9/00

(57) ABSTRACT

The present disclosure provides a method to treat or ameliorate vascular malformations, developmental abnormalities of one or more types of blood or lymphatic vessels. These are rare disorders with life-long risk of high morbidity including cosmetic concerns, pain, infection, pulmonary emboli, bleeding and even death. Treatment is difficult, and there is growing interest in improved therapies. The disclosure provides specific compounds that are inhibitors of Notch receptor signaling. In some embodiments, the compounds disclosed are gamma secretase inhibitors (GSIs).

### CROSS REFERENCE TO RELATED APPLICATIONS

This application is a § 371 U.S. National Stage of International Application PCT/US19/67062, filed Dec. 18, 2019 having Atty. Docket No. 150-31-PCT, which claims the benefit of U.S. Provisional Patent Application Ser. No. 62/781,473 filed Dec. 18, 2018, Julie Blatt, Atty. Dkt. 150-31-PROV, which is hereby incorporated by reference in its entirety.

### STATEMENT REGARDING FEDERALLY SPONSORED RESEARCH OR DEVELOPMENT

This invention was made with government support under Grant Numbers CA016086-40, ES010126-15A1, DK099156, and HL129086 awarded by the National Institutes of Health. The government has certain rights in the invention.

### 1. FIELD

The present disclosure provides a method to treat or ameliorate vascular malformations, developmental abnormalities of one or more types of blood or lymphatic vessels. These are rare disorders with life-long risk of high morbidity including cosmetic concerns, pain, infection, pulmonary emboli, bleeding and even death. Treatment is difficult, and there is growing interest in improved therapies. The disclosure provides specific compounds that are inhibitors of Notch receptor signaling. In some embodiments, the compounds disclosed are gamma secretase inhibitors (GSIs)

### 2. BACKGROUND

2.1. Introduction

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Vascular malformations are developmental abnormalities of venous, arterial, capillary or lymphatic vessels which grow with an individual. These can be purely of one type of vessel (e.g., venous [V] or lymphatic malformations [LM]) or can involve more than one type (e.g., venolymphatic malformations [VLM] or arteriovenous malformations [AVM]¹. Morbidities are significant such as pain, infection, pulmonary emboli, bleeding, cosmetic concerns, psychosocial issues, and even death. Current treatments include supportive care with compression garments, and interventions such as sclerotherapy, embolization, and surgical debulking or resection. There has been a growing interest in medical management of vascular malformations². The mammalian target of rapamycin (mTOR) inhibitor, rapamycin (Sirolimus), is the most commonly used drug, but is not effective for all vascular malformations, and generally does not result in their complete resolution³. While generally well-tolerated, its results often are not permanent, and life-long treatment may be needed. Thus, there is a need for other medications.

Notch proteins (Notch 1-4) are a family of receptors that are important in cell differentiation in virtually all tissues. The Notch pathway governs arteriovenous specification leading to distinction of arteries from veins⁴, and paradoxically both loss- or gain-of-function of Notch receptors have been linked to vascular malformations⁴. Ectopic Notch1⁵ or Notch4⁶⁻⁹ activation in endothelial cells results in AVMs. Importantly, activation of Notch4 in the endothelium of adult mice results in AVMs in organs, including liver, skin, uterus and brain⁷, and AVM formation was reversible upon loss of Notch4 transgene expression. Likewise, it has been shown that NOTCH1 signaling is activated in human brain AVMs^{9,10}. Knockdown of Notch1 in endothelial cells (ECs) has been found to decrease LEC proliferation and migration^{11,12}. These differing results may reflect tissue-, species-, and developmental stage-specific differences in the role of Notch signaling, but are all consistent with a role for Notch in aberrant angiogenesis. Several investigators have suggested targeting Notch as an approach to treating AVMs.

In mammals, the Notch signaling pathway is triggered by binding of Notch proteins to any of four activating ligands (Delta-like [Dll1, Dll4], Jagged [Jag1, Jag2]) which ultimately trigger transmembrane cleavage by a gamma-secretase complex, resulting in release of the Notch intracellular domain (NICD), which activates transcription of downstream target genes such as EphrinB2¹³. This is of particular interest because a number of GSIs have undergone phase I and II clinical trials in adults with Alzheimer's disease^{14,15} and in children and adults with cancer and related disorders¹⁶⁻¹⁸. That these drugs have known dosing and acceptable safety profiles makes them good candidates for drug repurposing for patients with other disorders including vascular malformations.

### 3. SUMMARY OF THE DISCLOSURE

The present disclosure provides a method of treating vascular malformations in a subject which comprises administering to the subject a Notch inhibitor. The vascular malformation may be a venous malformation (VM), a lymphatic malformation (LM), a venolymphatic malformation (VLM) or an arteriovenous malformation (AVM). The vascular malformation may be an extracranial vascular malformation or an intracranial vascular malformation.

In the embodiments above, the Notch inhibitor is a NOTCH 1, 2, 3 or 4 inhibitor. In some embodiments, the Notch inhibitor inhibits more than one Notch receptor protein. In other embodiments the Notch inhibitor is a gamma secretase inhibitor (GSI). In some embodiments, the Notch inhibitor is injected directly into a vascular malformation lesion. Alternatively, the Notch inhibitor may be delivered systemically. In other embodiments, the Notch inhibitor may be delivered topically.

The Notch inhibitor may be BMS-708163, BMS-906024, DAPT (GSI-IX), GSI 136, GSI-953, LY3039478, LY450139, MK-0752, NIC5-15, PF-03084014, or R04929097 or a pharmaceutically acceptable salt thereof.

In some embodiments, the subject may be a child. In other embodiments, the subject may be an adult.

This disclosure also provides a pharmaceutically acceptable formulation for the treatment of vascular malformations comprising a Notch inhibitor.

### 4. BRIEF DESCRIPTION OF THE FIGURES

FIG. 1A-1F. Internal elastic lamina is disorganized in brain and extra-cranial AVMs. (1A-1F) Representative images of normal arteries (1A and 1B) and arteriovenous malformations (1C-1F) stained with elastin in tissues from human brain (1C and 1D), and extra-cranial tissues: thigh (1E) and nose (1F). n=2-3 each group. Scale bars, 200 μm. Arrows point to internal elastic lamina (IEL) of major vessels stained with elastin showing either disorganization, diminished, or complete absence of the layer.

FIG. 2A-2F. Active NOTCH1 is aberrantly expressed in abnormal vascular channels. (2A-2F) Representative images of normal (N) and abnormal vascular channels (VC) stained with smooth muscle marker (αSMA) and Notch1 intracellular domain (NOTCH1-ICD) in tissues from human lymphatic malformation (LM, FIG. 2A-2C) and arteriovenous malformation (AVM, FIG. 2D-2F). n=2 each group. Scale bar, 100 μm. Arrows point to endothelial lining of vessels prominently expressing NOTCH1-ICD. Boxed regions are shown as digitally zoomed insets to the right.

FIG. 3A-3H. NOTCH2 and 3 are prominently expressed in the endothelial and some mural cells lining venous and lymphatic malformations. (3A-3H) Representative images of NOTCH2 (3A-3D) and NOTCH 3 (3E-3H) in human extra-cranial venous (VM) and lymphatic malformations (LM). n=2-4 each group. 3B, 3D, and 3F, 3H represent digitally zoomed image of boxed insets in 3A, 3C and 3E, 3G respectively. Dotted red lines highlight regions of NOTCH2 and 3 expression in mural cells surrounding malformations. Scale bar, ˜200 μm. Arrows point to the endothelial and mural cells lining malformed vessels prominently expressing NOTCH2 and 3. “N” represents a normal vessel within the same section.

FIG. 4A-4F. NOTCH4-ICD shows irregular expression in abnormal vascular channels. (4A-4F) Representative images of abnormal vascular channels (VC) stained with endothelial marker (CD31) and NOTCH4-ICD in tissues from human lymphatic malformation (LM, FIGS. 4A and 4D), arteriovenous malformation (AVM, FIGS. 4B and 4E), and venous malformation (VM, FIGS. 4C and 4F). n=2 each group. Scale bar, 100 μm. Arrows point to endothelial lining of vessels showing the presence or absence of NOTCH4-ICD.

FIG. 5A-5D. DAFT and RO4929097 downregulate downstream Notch target Hey1 without altering cell viability. (5A, 5B) Measurement of HUVEC (5A) and hLEC (5B) cell viability after GSI treatments. Quantitative data are represented as mean+SEM, n=3 for HUVEC and hLEC. Significance was determined by 2-tailed, type 2 Student's t test, *P<0.05. (5C, 5D) Relative expression of downstream Notch target gene Hey1 in GSI-treated HUVEC (5C) and hLEC (5D). Quantitative data are represented as mean values of fold change over DMSO control±SEM. n=4 for each cell line. Gapdh and β-actin were used as housekeeping control. Significance was determined by 2-tailed, type 2 Student's t test, *P<0.05, **P<0.01.

FIG. 6A-6D. GSIs block cellular migration (6A, 6B) Control DMSO or GSI treated hLEC (6A) at 72 hrs and HUVEC (6B) at 24 hrs post-scratch. (6C, 6D) Migration from the time of scratch (T=0) for hLEC (6C) and HUVEC (6D) was measured. Quantitative data are represented as mean±SEM. n=4 for HUVEC and n=3 for hLEC. Significance was determined by 2-tailed, type 2 Student's t test, *P<0.05, **P<0.01. Scale bar, 200 μM.

FIG. 7A-7D. GSIs block tube formation (7A, 7B) Representative images of tubes formed by control DMSO treated or GSI DAFT (7A) or RO4929097 (7B) treated HUVEC at 24 hrs. (7C, 7D) Branch points per field were quantified. Quantitative data are represented as mean±SEM. n=4 for DAPT treated (7C) or RO treated (7D) HUVEC. Significance was determined by 2-tailed, type 2 Student's t test, ***P<0.001. Scale bar, 100 μM.

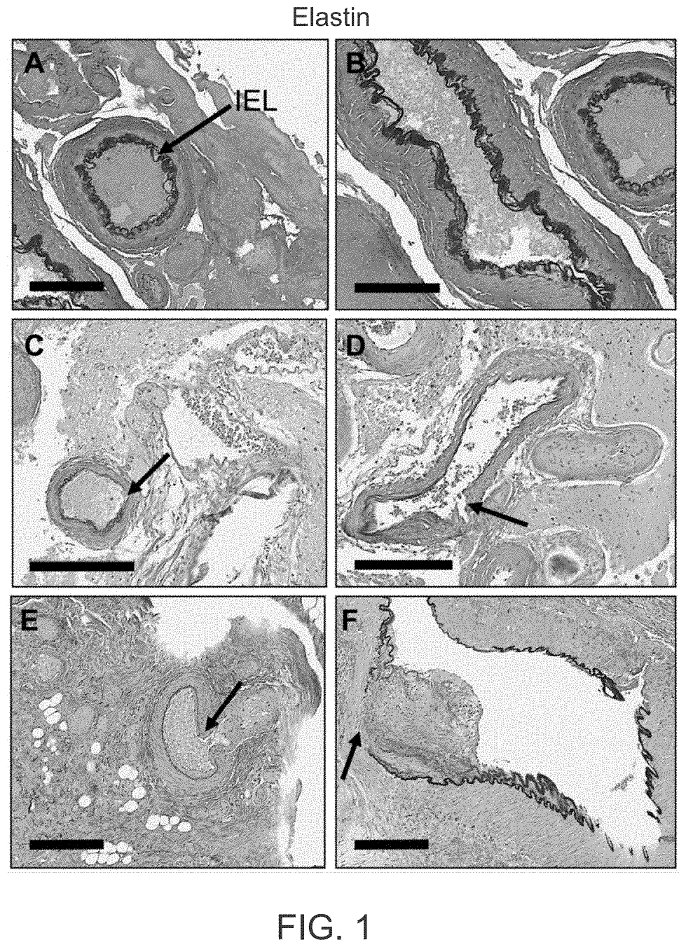

Supplemental FIG. 1A-1D. Vascular NOTCH4 expression in control and vascular malformation tissues. 1A, 1B) NOTCH4 is expressed in CD31+ endothelial cells in control neonatal skin. 1A) Notch4 antibody, 1B) no primary antibody. White arrows marker the NOTCH4 expressing vessels. White arrowheads mark absence of NOTCH4 expression. 1C, 1D) NOTCH4 expression is variable in the CD31+ endothelial of vascular malformations. 1C) VM, D) LM. White arrows marker the NOTCH4 expressing vessels. White arrowheads mark absence of NOTCH4 expression. Asterisk marks normal artery in LM section. Scale bar 50 microns.

### 5. DETAILED DESCRIPTION OF THE DISCLOSURE

In this study, we show that extracranial vascular malformations, like brain AVMs, are characterized by diminished and incomplete smooth muscle actin (αSMA) in the vascular smooth muscle cells (VSMC) of AVM, and diminished elastin coverage in the internal elastin lamina (IEL) of AVMs. NOTCH1-4 proteins are variably expressed in a range of these extracranial vascular malformations involving lymphatic, venous and arterial vessels. Moreover, two GSIs-DAPT (GSI-IX) and RO1909297—caused dose-dependent inhibition of Notch target gene expression (Hey1) and angiogenesis of both lymphatic and blood endothelial cells in vitro. These results provide additional rationale to support clinical trials of GSIs in patients with vascular malformations.

Notch expression has been shown to be aberrant in brain AVMs and targeting Notch has been suggested as an approach to their treatment. It is unclear whether extracranial vascular malformations follow the same patterning and Notch pathway defects. In this study, we examined human extracranial VM (n=3), LM (n=10), and AV (n=6) malformations, as well as sporadic (non-syndromic) brain AVMs (n=3). In addition to showing that extracranial AVMs demonstrate interrupted elastin and that AVMs and LMs demonstrate abnormal α-smooth muscle actin just as brain AVMS do, our results demonstrate that NOTCH1, 2, 3 and 4 proteins are overexpressed to varying degrees in both the endothelial and mural lining of the malformed vessels in all types of malformations, although not necessarily in every malformation of each type. We further show that two GSIs, DAPT and RO1909297, cause dose-dependent inhibition of Notch target gene expression (hey1) and rate of migration of monolayer cultures of human lymphatic endothelial cells (hLECs) and blood endothelial cells (HUVEC). GSIs also inhibit HUVEC network formation. hLECs are more sensitive to GSIs compared to HUVEC. GSIs have been found to be relatively safe in clinical trials in patients with Alzheimer's disease or cancer. Our results support the use of Notch inhibitors in patients with vascular malformations.

5.1. Definitions

While the following terms are believed to be well understood by one of ordinary skill in the art, the following definitions are set forth to facilitate explanation of the presently disclosed subject matter.

As used herein the phrase “gamma secretase inhibitor” (GSI) means a compound capable of inhibiting the mammalian gamma secretase enzyme complex that cleaves various transmembrane proteins including amyloid precursor protein (APP). See Gu et al., 2017, “Gamma secretase inhibitors: a patent review” Expert Opinion on Ther. Patents 27(7) 851-866. Examples of GSIs are BMS-708163 ((2R)-2-(N-(2-fluoro-4-(1,2,4-oxadiazol-3-yl)benzyl)-4-chlorophenylsulfonamido)-5,5,5-trifluoropentanamide, avagacestat, PCT Pub. No. WO2014/177915); BMS-906024 ((2R,3S)-N-[(3S)-1-Methyl-2-oxo-5-phenyl-2,3-dihydro-1H-1,4-benzodiazepin-3-yl]-2,3-bis(3,3,3-trifluoropropyl)succinimide, PCT Pub. No. WO 2012/129353); DAPT (GSI-IX) (N-[N-(3,5-difluorophenacetyl-L-alanyl)]-S-phenylglycine t-butyl ester, U.S. Pat. No. 8,853,274); GSI-136 (5-chloro-N-[(2S)-3-ethyl-1-hydroxypentan-2-yl]thiophene-2-sulfonamide, U.S. Pat. Nos. 6,878,742, 7,691,884, 7,842,718); GSI-953 (5-chloro-N-[(1S)-3,3,3-trifluoro-1-(hydroxymethyl)-2-(trifluoromethyl)propyl]thiophene-2-sulfonamide, begacestat, PCT Pub. No. WO2004/092155); LY3039478 (4,4,4-Trifluoro-N-((S)-1-(((S)-5-(2-hydroxyethyl)-6-oxo-6,7-dihydro-5H-benzo[d]pyrido[2,3-b]azepin-7-yl)amino)-1-oxopropan-2-yl)butanamide, crenigacestat, Bhagat et al., 2017 J. Biol Chem 292(3) 837-846, Massard et al., Ann Oncol 29(9) 1911-1917); LY450139 ((2S)-2-hydroxy-3-methyl-N-[(2S)-1-[[(5S)-3-methyl-4-oxo-2,5-dihydro-1H-3-benzazepin-5-yl]amino]-1-oxopropan-2-yl]butanamide, semagacestat, US Pat. Pub. No. 2011/105471); MK-0752 (3-{cis-4-[(4-Chlorophenyl)sulfonyl]-4-2,5-difluorophenyl)cyclohexyl}propanoic acid, U.S. Pat. No. 8,853,274); NIC5-15 (1S,2S,4S,5R)-6-methoxycyclohexane-1,2,3,4,5-pentol, d-pinatol , 3-O-methyl-D-chiro-inositol, D-(+)-chiro-inositol, D-pinitol, inzitol, D-(+)-pinitol, (+)-pinitol, sennitol, pinitol, (+/−)pinitol); PF-03084014 (N²-[(2S)-6,8-Difluoro-1,2,3,4-tetrahydro-2-naphthalenyl]-N-(1-{1-[(2,2-dimethylpropyl)amino]-2-methyl-2-propanyl}-1H-imidazol-4-yl)-L-norvalinamide, nirogacestat, PCT Pub. No. WO2007/0034326); or RO4929097 (2,2-Dimethyl-N-[(7S)-6-oxo-6,7-dihydro-5H-dibenzo[b,d]azepin-7-yl]-N′-(2,2,3,3,3-pentafluoropropyl)malonamide, US Pat. Pub. No. 2014/0357620). The GSI may be a dipeptide analogue such as DAPT, DBZ ((S)-2-(2-(3,5-difluorophenyl)acetamido)-N-((S)-5-methyl-6-oxo-6,7-dihydro-5H-dibenzo[b,d]azepin-7-yl)propenamide, YO-01027, US Pat. Pub. No. 2013/196976), LY450139, or PF3084014. Alternatively, it may contain a benzodiazepine backbone structure such as BMS-906024 or RO4929097. In other embodiments, the GSI may contain a sulfonamide structure such as BMS-299897 (4-{2-[(1R)-1-{[(4-Chloropheny)sulfonyl](2,5-difluorophenyl)amino}ethyl]-5-fluorophenyl}butanoic acid, US Pat. Pub. No. 2012/835508), BMS-708163 or GSI-953. The GSI may be deuterated using methods known to those skilled in the art, see below.

As used herein a “Notch inhibitor” is a compound that modulates the signaling from a Notch receptor protein. The Notch receptor protein may be NOTCH 1, NOTCH 2, NOTCH 3 or NOTCH 4. NOTCH 1 is also known as Neurogenic locus notch homolog protein 1 with UniProtKB/Swiss-Prot accession no. P46531.4; a 2555 aa linear protein, NOTC1_HUMAN, updated 7 Nov. 2018 or neurogenic locus notch homolog protein 1 preproprotein [Homo sapiens], NCBI Reference Sequence: NP_060087.3; a 2555 aa linear protein NP _060087 updated 25 Nov. 2018. NOTCH 2 is also known as Neurogenic locus notch homolog protein 2 with UniProtKB/Swiss-Prot accession no. Q04721.3; a 2471 aa linear protein, NOTC2_HUMAN updated 7 Nov. 2018 or neurogenic locus notch homolog protein 2 isoform 1 preproprotein [Homo sapiens], NCBI Reference Sequence: NP_077719.2; a 2471 aa linear protein, NP_077719 updated 23 Nov. 2018. NOTCH 3 is also known as Neurogenic locus notch homolog protein 3 with UniProtKB/Swiss-Prot accession no. Q9UM47.2; a 2321 aa linear protein, NOTC3_HUMAN updated 7 Nov. 2018 or neurogenic locus notch homolog protein 3 precursor [Homo sapiens] NCBI Reference Sequence: NP_000426.2; a 2321 aa linear protein NP_000426, updated 22 Nov. 2018. NOTCH 4 is also known as Neurogenic locus notch homolog protein 4 with UniProtKB/Swiss-Prot accession no. Q99466.2; a 2003 aa linear protein, NOTC4_HUMAN updated 7 Nov. 2018 or neurogenic locus notch homolog protein 4 preproprotein [Homo sapiens], NCBI Reference Sequence: NP_004548.3; a 2003 aa linear protein. NP_004548 updated 22 Nov. 2018. The “Notch inhibitor” may be a monoclonal antibody such as demcizumab (OMP21M18), navicixizumab (OMP-305B8, bronticuzumab (OMP52M51), tarextumab (OMP-59R5) or enoticumab (REGN421), See Columbo et al., 2015, Oncotarget 6(29) 26826-26840; Lamy et al., 2017, New Biotechnology 39 215-221; and Venkatesh et al., 2018, Stem Cell Invest 5, 5 p. 1-12.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to any of a diluent, adjuvant, excipient or carrier with which at least one compound of the present disclosure is administered. “Subject” includes mammals and humans. The terms “human” and “subject” are used interchangeably herein.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, the mode of administration, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., improvement or stabilization of a discernible symptom), physiologically, (e.g., improvement or stabilization of a physical parameter), or both, or inhibiting at least one physical parameter which may not be discernible to the subject.

As used herein the term “vascular malformations” are developmental abnormalities of the vessels that carry the blood or lymph. They may be anomalies of one type of vessel (e.g., capillary [CM], venous [VM] or lymphatic malformations [LM]), or can involve more than one type (e.g., venous and lymphatic vessels, veno-lymphatic malformations [VLM] or arterial and venous vessels, arteriovenous malformations [AVM]).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Substitution with positron emitting isotopes, such as ¹¹C, ¹⁸F, ¹⁵O and ¹³N, can be useful in Positron Emission Topography (PET) studies.

Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents. In another embodiment, the isotope-labeled compounds contain deuterium (²H), tritium (³H) or ¹⁴C isotopes. Isotope-labeled compounds of this invention can be prepared by the general methods well known to persons having ordinary skill in the art.

Such isotope-labeled compounds can be conveniently prepared by carrying out the procedures disclosed in the Examples disclosed herein by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O. Alternatively, deuterium may be also incorporated into a compound using methods such as through reduction such as using LiAlD₄ or NaBD₃, catalytic hydrogenation or acidic or basic isotopic exchange using appropriate deuterated reagents such as deuterides, D₂ and D₂O. In addition to the above, PCT publications, WO2014/169280; WO2015/058067; U.S. Pat. Nos. 8,354,557; 8,704,001 and US Patent Application Publication Nos.; 2010/0331540; 2014/0081019; 2014/0341994; 2015/0299166, the methods are hereby incorporated by reference.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Alternatively, depending on the context, the term “about” may mean + one half a standard deviation, + one standard deviation, or ± two standard deviations. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the verb “comprise” as used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Antibodies

Another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. Alternatively, monomeric binders such as scFv, diabodies, minibodies, small immunoproteins (SIPs) may be prepared. Olafsen et al. 2005 Cancer Res 65:5907-5916; Borsi et al. 2002 Int J Cancer 102:75-85; Berndorff et al. 2005 Clin Cancer Res 11:7053s-7063s; and Tijink et al. 2006 J Nucl Med 47:1127-1135. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Antibody-producing cells can be obtained from the subject and used to prepare monoclonal antibodies by standard techniques, such as the hybridoma technique originally described by Kohler and Milstein 1975 Nature 256:495-497, the human B cell hybridoma technique (see Kozbor et al., 1983, Imnunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409 (Winter); PCT Publication Nos. WO 92/18619; WO 91/17271; WO 92/20791; WO 92/15679; WO 93/01288; WO 92/01047; WO 92/09690; WO 90/02809; Fuchs et al. 1991 Bio/Technology 9:1370-1372; Hay et al. 1992 Hum. Antibod. Hybridomas 3:81-85; Huse et al. 1989 Science 246:1215-1281; Griffiths et al. 1993 EMBO J. 12:725-734.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 5,225,539 (Winter); U.S. Pat. No. 4,816,567 (Cabilly et al.); European Patent Application 125,023; Better et al. 1988 Science 240:1041-1043; Liu et al. 1987 Proc. Natl. Acad. Sci. USA 84:3439-3443; Liu et al. 1987 J. Immunol. 139:3521-3526; Sun et al. 1987 Proc. Natl. Acad. Sci. USA 84:214-218; Nishimura et al. 1987 Cancer Res. 47:999-1005; Wood et al. 1985 Nature 314:446-449; and Shaw et al. 1988 J. Natl. Cancer Inst. 80:1553-1559; Morrison 1985 Science 229:1202-1207; Oi et al. 1986 Bio/Techniques 4:214; Jones et al. (1986) Nature 321:552-525; Verhoeyen et al. 1988 Science 239:1534-1536; and Beidler et al. 1988 J. Immunol. 141:4053-4060.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806 (Lonberg el al.). In addition, companies such as Abgenix, Inc. (Freemont, Calif.), can be engaged to provide human antibodies directed against a selected antigen using technology similar to that described above.

Nelson et al. and Nieri et al. recently reviewed therapeutic antibodies either on the market or in clinical development, and current techniques for their production. Nelson et al. 2010 Nat Rev Drug Disc 9 767-774; Nieri et al. 2009 Curr Top Med Chem 16 753-779.

Fully human antibodies also may be produced via CHO cell culture and by transgenic animals and plants. Full-size human monoclonal antibodies are now extracted by milk of transgenic animals (e.g., cows, goats). Redwan 2009 J Immunoass Immunochem 30 262-290. Also plants, like tobacco, are used for making antibodies. Tobacco is relatively easy to transfect using the tobacco virus. Yusibov et al. 2011 Hum Vacc 7(3) 313-321.

This technology is particularly well suited to modifying Fc-fusion proteins. Carter 2011 Exp. Cell Res 317 1261-1269; Czajkowsky et al. 2012 EMBO Mol Med 4 1015-1028.

5.2. Pharmaceutically Acceptable Compositions

Provided herein are pharmaceutical compositions comprising a compound disclosed herein as an active ingredient, or a pharmaceutically acceptable salt, solvate or hydrate thereof in combination with a pharmaceutically acceptable vehicle, carrier, diluent, or excipient, or a mixture thereof.

The compound provided herein may be administered alone, or in combination with one or more other compounds provided herein. The pharmaceutical compositions that comprise a compound disclosed herein can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, 21st Ed., Lippincott, Williams & Wilkins, Baltimore, Md., 2006; Modified-Release Drug Delivery Technology, Rathbone et al., Eds., Drugs and the Pharmaceutical Science, Marcel Dekker, Inc.: New York, N.Y., 2003; Vol. 126).

In one embodiment, the pharmaceutical compositions are provided in a dosage form for oral administration, which comprise a compound provided herein, e.g., a compound disclosed herein or a pharmaceutically acceptable salt, solvate or hydrate thereof; and one or more pharmaceutically acceptable excipients or carriers.

In another embodiment, the pharmaceutical compositions are provided in a dosage form for parenteral administration, which comprise a compound disclosed herein or a pharmaceutically acceptable salt, solvate or hydrate thereof; and one or more pharmaceutically acceptable excipients or carriers.

In yet another embodiment, the pharmaceutical compositions are provided in a dosage form for topical administration, which comprise a compound disclosed herein or a pharmaceutically acceptable salt, solvate or hydrate thereof; and one or more pharmaceutically acceptable excipients or carriers.

The pharmaceutical compositions provided herein can be provided in a unit-dosage form or multiple-dosage form. A unit-dosage form, as used herein, refers to a physically discrete unit suitable for administration to a human or animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons. The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In one embodiment, the therapeutically, effective dose is from about 0.1 mg to about 2,000 mg per day of a compound provided herein. The pharmaceutical compositions therefore should provide a dosage of from about 0.1 mg to about 2000 mg of the compound. In certain embodiments, pharmaceutical dosage unit forms are prepared to provide from about 1 mg to about 2000 mg, from about 10 mg to about 1000 mg, from about 20 mg to about 500 mg or from about 25 mg to about 250 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

5.2.1. Parental Administration

The pharmaceutical compositions provided herein can be administered parenterally by injection, infusion, or implantation, for local or systemic administration. Parenteral administration, as used herein, include intravenous, intralesional administration, intraarterial, intraperitoneal, intrathecal, intraventricular, intraurethral, intrasternal, intracranial, intramuscular, intrasynovial, intravesical, and subcutaneous administration.

The pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfate and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein can be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In one embodiment, the lyophilized nanoparticles are provided in a vial for reconstitution with a sterile aqueous solution just prior to injection. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions. The pharmaceutical compositions provided herein can be formulated as immediate or modified release dosage forms, including delayed-, sustained, pulsed-, controlled, targeted-, and programmed-release forms.

The pharmaceutical compositions can be formulated as a suspension, solid, semi-solid, or thixotropic liquid, for administration as an implanted depot.

5.2.2. Oral Administration Compositions

The pharmaceutical compositions provided herein can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, troches, lozenges, pastilles, cachets, pellets, medicated chewing gum, bulk powders, effervescent or non-effervescent powders or granules, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, Bhatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSILA® 200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Hydrophilic polymer formulations have been widely used for improved oral availability such as ethylene oxides, hydroxy propyl methyl cellulose (HPC), polyethylene oxide) (PEO), polyvinyl alcohol (PVA), poly(hydroxyethylmethyl acrylate) methyl methacrylate (PHEMA), or vinyl acetate (PCT Pub. No. WO1999/37302 (Alvarez et al.); Dimitrov & Lambov, 1999, Int J Pharm 189 105-111; Zhang et al., 1990, Proc Int. Symp Controlled Release Bioact. Mater. 17, 333, the contents of which are hereby incorporated by reference in their entirety). Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545, the contents of which are hereby incorporated by reference in their entirety. The capsules may also be coated as known by those of skill in the art in order to modify or sustain dissolution of the active ingredient.

The pharmaceutical compositions provided herein can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458, the content of which is hereby incorporated by reference in its entirety.

The pharmaceutical compositions provided herein can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms. The pharmaceutical compositions provided herein can be formulated as immediate or modified release dosage forms, including delayed-, sustained, pulsed-, controlled, targeted-, and programmed-release forms.

The pharmaceutical compositions provided herein can be co-formulated with other active ingredients which do not impair the desired therapeutic action, or with substances that supplement the desired action.

5.2.3. Topical Administration

The pharmaceutical compositions provided herein can be administered topically to the skin, orifices, or mucosa. The topical administration, as used herein, includes dermal application, (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

Efficacy of topical application of antiangiogenic agents: Rapamycin is commercially available as a pill or liquid. It has been compounded for application to the skin, and successfully used for many years in that format in the treatment of tuberous sclerosis. See Malissen et al., 2017, Long-term treatment of cutaneous manifestations of tuberous sclerosis complex with topical 1% sirolimus cream: A prospective study of 25 patients. Am Acad Dermatol. 77:464-472.e3; and Greveling et al., 2017, Treatment of port wine stains using Pulsed Dye Laser, Erbium YAG Laser, and topical rapamycin(sirolimus)-A randomized controlled trial. Lasers Surg Med. 49(104-109. Recent data show that rapamycin can be applied to the skin of patients with superficial LMs or non-syndromic cutaneous CMs with excellent clinical responses. See Garcia-Montero et al., 2017, Microcystic Lymphatic Malformation Successfully Treated With Topical Rapamycin. Pediatrics. 139(4):e20162105; and Ivars M, Redondo P., 2017, Efficacy of Topical Sirolimus (Rapamycin) for the Treatment of Microcystic Lymphatic Malformations. JAMA Dermatol. 153:103-105. Perhaps the best known effective use of oral agents formulated for skin application in the treatment of vascular anomalies is the use of timolol (topical propranolol) for the treatment of cutaneous infantile hemangiomas. See Léauté-Labréze C, Hoeger P, Mazereeuw-Hautier J, et al. A randomized, controlled trial of oral propranolol in infantile hemangioma. N Engl J Med. 2015; 372:735-746. For both propranolol and sirolimus, topical efficacy has correlated with systemic efficacy for similar lesions.

5.3. Aerosol Administration

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. There are many examples in the literature of metered dose inhalers (MDIs) or pressurized metered dose inhalers (pMDIs). See Kleinstreuer et al. 2015 World J Clin Cases 2(12) 742-756. The pharmaceutical compositions can also be provided as a dry powder for insufflation, alone or in combination with an inert carrier such as lactose or phospholipids; and nasal drops. Many pulmonary drugs are delivered by dry powder inhalers (DPIs) with differing arrangements such as single dose, multi-dose with the pharmaceutical composition in bulk, or multi-dose with individual blister packs. See Kleinstreuer et al. 2015; Weer and Miller 2015 J Pharm Sci 104 3259-3288. For intranasal use, the powder can comprise a bioadhesive agent, including chitosan or cyclodextrin. As mentioned above, the pharmaceutical composition may be delivered by nebulizer such as an atomizer (jet nebulizer), an ultrasonic wave nebulizer, or a vibrating mesh nebulizer. See Kleinstreuer et al. 2015.

Alternatively, the pharmaceutical composition may be dissolved in glycerol, propane 1,2 diol gycol (PG), water or a mixture thereof and vaporized at relatively low temperature (>100° C., typically 40-65 oC) in an e-cigarette. See Bertholon et al. 2013 Respiration 86 433-438; Brown and Cheng 2014 Tob Control May; 23 Suppl 2:ii4-10; and Famele 2015 Nicotine Tob Res 271-279; European Patent Appn. No. EP2641490A1 (Liu); European Patent Nos. EP1618803B1 and EP1736065B1 (Hon L., Best Partners Worldwide Limited); U.S. Appn. Nos. 20050016550 A1 (Katase), 20110265806 (Alarcon and Healy), 20110277780A1 (Terry and Minskoff), 20130213418 A1 (Tucker et al., Altria Client Service Inc.), 20130192621 (Li et al, Altria Client Service Inc.), 20130192623 (Tucker et al., Altria Client Service Inc.), 20130213419 (Tucker et al., Altria Client Service Inc.), 20130220315 (Conley, Fuma International); U.S. Pat. No. 8,490,628 (Hon, Ruyan Investment Limited), U.S. Pat. No. 8,528,569 (Newton), U.S. Pat. No. 8,550,069 (Alelov).

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions provided herein for inhaled/intranasal administration can further comprise a suitable flavor, such as menthol and levomenthol, or sweeteners, such as saccharin or saccharin sodium,

The pharmaceutical compositions provided herein for topical administration can be formulated to be immediate release or modified release, including delayed-, sustained-, pulsed-, controlled-, targeted, and programmed release.

5.4. Intralesional Injection

Endovascular therapy, consisting mainly of intralesional sclerosant injection, is an accepted treatment for vascular malformations (Burrows, 2013, Tech Vasc Interv Radiol 16, 12-21; Myers 2018, Phlebology doi: 10.1177/0268355518798283). This is usually carried out under general anesthesia or deep sedation. Injection catheters for directing placement of sclerosants are introduced under ultrasound visualization. Sclerosants for VM include ethanol, 3% sodium tetradecyl sulfate, and bleomycin. LM can be injected with doxycycline, bleomycin, OK-432, or other sclerosants. Although most vascular malformations are not cured, the majority of patients benefit from endovascular treatment. Complications of sclerotherapy vary with the sclerosing agent but include both systemic and local effects. Although the toxicities are minimized with increasing experience and observation of maximum doses, disadvantages of ethanol include potential severe systemic effects (intoxication, hypoglycemia, cardiac arrhythmias, and cardiovascular collapse) and an increased rate of severe local complications (e.g., skin necrosis and neuropathy) compared with detergent sclerosants. Foamed detergents such as sodium tetradecyl sulfate are as effective as ethanol in controlling the patients' symptoms and have a significantly reduced rate of systemic complications and neuropathy, compared with ethanol. Staged treatment with foam sclerosant is currently used by the majority of practitioners in North America and Europe for VMs. Some practitioners use foam for most VMs, reserving ethanol for lesions that recurred or did not respond to a foam sclerosant. Bleomycin is effective in areas that are sensitive to swelling, such as the oral cavity and orbit, but the potential risk of pulmonary fibrosis or acute lung reaction must be taken into consideration.

Intralesional therapies targeted at genetic components of VMs have not been used. These would have a theoretical advantage over systemic administration of the same agents since systemic absorption and toxicities would likely be minimized. Thus, it should be possible to administer lower doses when these are introduced intralesionally. These concepts are supported by the use of intralesional bleomycin which has rarely been reported to cause lung toxicity, in contrast to when it is administered systemically. Targeted intralesional treatments such as GSIs also should be more effective than the sclerosants discussed above, since their effects would be directly on the malformation and not indirectly through generating local inflammation.

5.5. Modified Release Formulations

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500, the contents of which are hereby incorporated by reference in their entirety.

5.5.1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethylene terephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed; the polymer viscosity; the particle sizes of the polymer and/or the active ingredient(s); the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, melt-granulation followed by compression.

5.5.2. Osmotic Controlled Release Devices

The pharmaceutical compositions provided herein in a modified release dosage form can be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, potyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220, the contents of which are hereby incorporated by reference in their entirety.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the att. See, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27, the contents of which are hereby incorporated by reference in their entirety.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918, the contents of which are hereby incorporated by reference in their entirety. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxyl ethyl cellulose, and other pharmaceutically acceptable excipients or carriers.

5.5.3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

5.6. Dosage

The pharmaceutical compositions that are provided can be administered for prophylactic and/or therapeutic treatments. An “effective amount” refers generally to an amount that is a sufficient, but non-toxic, amount of the active ingredient (i.e., a compound disclosed herein) to achieve the desired effect, which is a reduction or elimination in the severity and/or frequency of symptoms and/or improvement or remediation of damage. A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom.

In general, toxicity and therapeutic efficacy of the compound disclosed herein can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In many cases data obtained from cell culture and/or animal studies can be used in formulating a range of dosages for humans. The dosage of the active ingredient typically lines within a range of circulating concentrations that include the ED50 with little or no toxicity. The dosage can vary within this range depending upon the dosage form employed and the route of administration utilized.

The effective amount of a pharmaceutical composition comprising a compound disclosed herein to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the compound disclosed herein is being used, the route of administration, and the size (body weight, body surface or organ size) and/or condition (the age and general health) of the patient. A clinician may modify the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 150 mg/kg; or 1 μg/kg up to about 100 mg/kg; or 5 μg/kg up to about 50 mg/kg.

The dosing frequency will depend upon the pharmacokinetic parameters of the compound disclosed herein in the formulation. For example, a clinician will administer the composition until a dosage is reached that achieves the desired effect. The composition may therefore be administered as a single dose or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 6. EXAMPLES

Extracranial Vascular Malformations Have Discontinuous Elastin Fibers in Their Internal Elastic Lamina.

Human brain AVMs have disrupted elastin fibers, which compromise the structural integrity of the vessel and mediate arteriovenous malformations¹⁹⁻²¹. While brain elastin content has been well characterized, the elastin in extracranial vascular malformation has not been studied. Therefore, we evaluated tissue samples from human extracranial AVM for elastin fibers in the IEL. Compared to control vessels (FIGS. 1A and 1B), both brain (FIGS. 1C and 1D) and extra-cranial AVMs (FIGS. 1E and 1F) displayed diminished and discontinuous elastin staining in the IEL of major vessels. An additional 3 AVM samples obtained from different regions of the body showed interrupted lamina similar to FIG. 1C-1F. Our results indicate that IEL elastin levels are not only abnormal in brain AVMs, but also in extracranial AVMs.

Active NOTCH1 is Expressed in Abnormal Human Extracranial Vascular Channels.

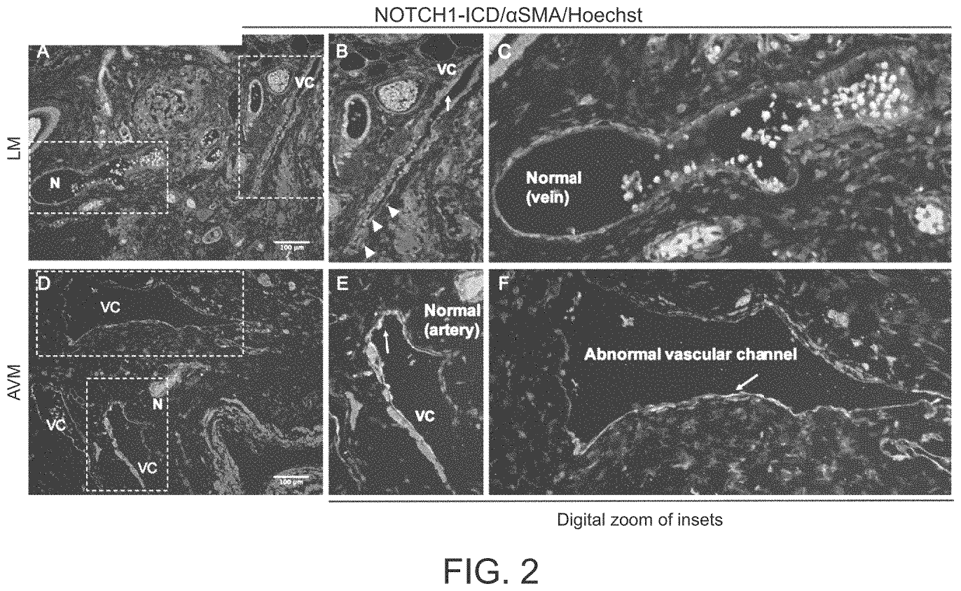

Normal arteries have a uniform layer of αSMA-positive mural cells around them, and in contrast, normal lymphatic capillaries have a single endothelial layer with no mural cell coverage. However, arteries involved in AVMs are characterized by incomplete αSMA coverage and malformed lymphatic vessels gain mural cell coverage around them²² (FIG. 2A-2F). Due to the role of Notch signaling in normal and pathological vascular development and arterial venous specification, we determined the expression of active NOTCH1 intracellular domain (NOTCH1-ICD) in abnormal vascular channels (VC). Compared to minimal expression in normal (N) vessels with complete αSMA (FIG. 2C), NOTCH1-ICD was clearly seen in all the abnormal VC (FIG. 2A, 2B, 2D-2F) within the same sections. There was no NOTCH1-ICD expression overlapping with αSMA staining indicating that NOTCH1 is primarily activated in the endothelium of malformed vessels. An additional 8 tissues from a broad spectrum of LM subtypes were stained with an antibody against the extracellular domain of NOTCH1, and the lymphatic endothelial protein, PODOPLANIN (The organ/tissue for the samples are listed in Supplementary Table 1, the staining results are in Supplemental Table 2). NOTCH1 expression was observed in the endothelium of 6 of these LM samples, but not in normal lymphatic vessels.

Notch2 and 3 are expressed in endothelial and mural cells surrounding vascular malformations.

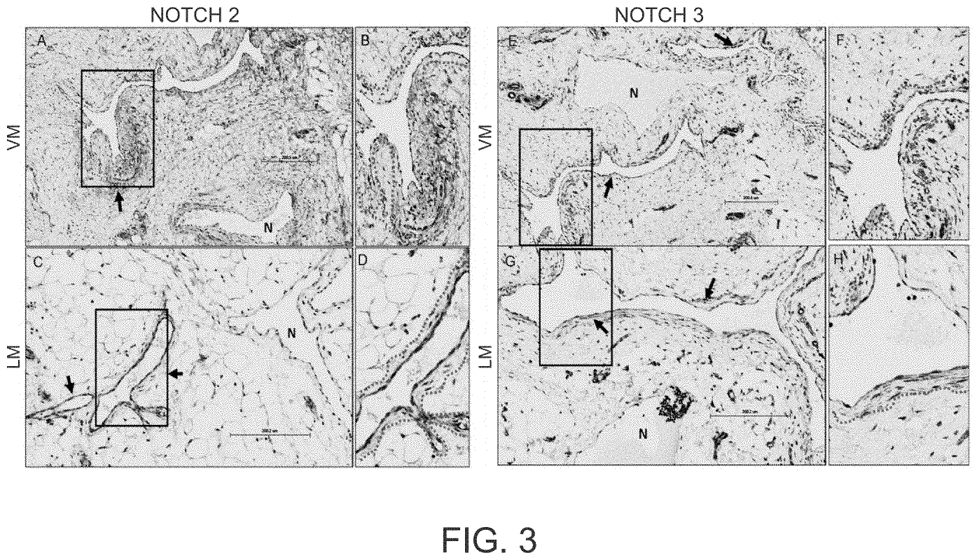

Studies have shown that NOTCH2 is minimally expressed, while NOTCH3 is overexpressed in human brain vascular malformations and a combined loss of one allele of Notch1 with Notch3 deletion results in CADASIL (a genetically mediated arteriopathy primarily of cerebral arteries and arterioles) and AVMs in mice^{21,23}. Therefore, we examined NOTCH2 and NOTCH3 expression in extracranial LM and VM. Our results show NOTCH2 and NOTCH3 clearly are expressed in endothelial and mural cells lining both VMs and LMs, and appear to be increased compared to normal vessels (N) within the same field (FIG. 3A-3D). NOTCH2 was expressed in cells outside of the vasculature in venous malformations and it was distinctly expressed mainly in vascular channels of LMs (FIGS. 3A and 3C). NOTCH3 staining was obvious in both LM and VM (FIGS. 3B and 3D). Our results suggest that unlike in brain AVMs, NOTCH2 and NOTCH3 are expressed in extracranial lymphatic and venous malformations, and in the endothelial and to some extent even mural cell layer surrounding the malformations.

Notch4 Shows Variable Expression in Malformed Vessels of Venous, Lymphatic and Arteriovenous Origin.

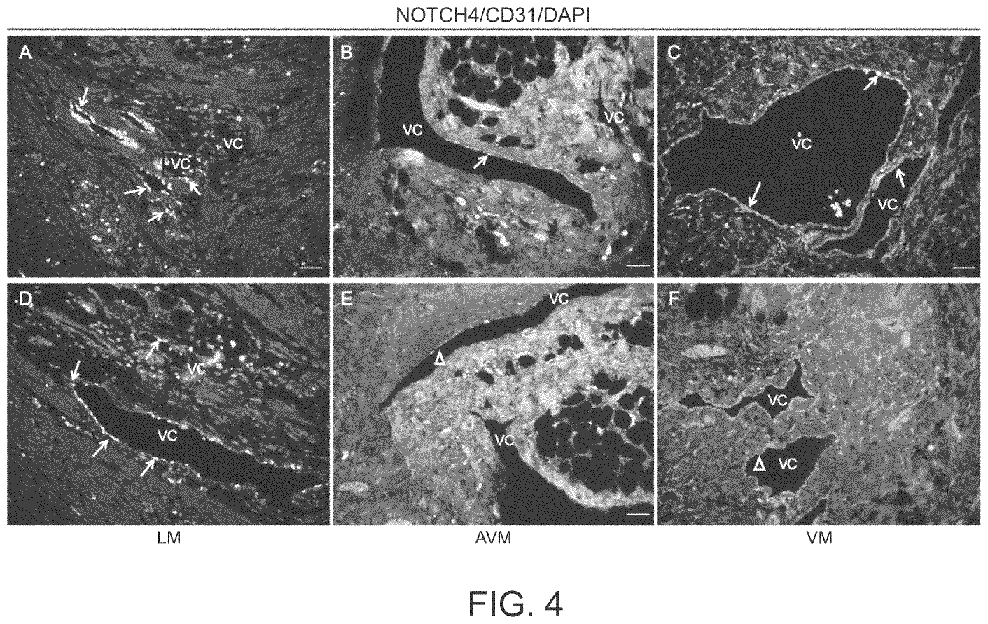

Staining with an antibody against NOTCH4-intracellular domain (NOTCH4-ICD) demonstrated varied expression across different types of malformations. Lymphatic malformation sections showed strong NOTCH4-ICD expression in CD31+ endothelial cells lining the abnormal vascular channels consistent with active NOTCH4-ICD signaling (FIG. 4A, 4D). NOTCH4-ICD expression in AVM sections was minimal with occasional high expression in a few endothelial cells (FIG. 4B, 4E). Tissue from VM showed weak and interrupted NOTCH4-ICD expression in CD31 weak vessels and was absent from CD31+ abnormal vascular channels (FIG. 4C, 4F). Collectively, these data suggest a link between the misexpression of Notch family members and NOTCH1 and NOTCH4 activation in human extracranial vascular malformations of all types.

DAPT and RO4909297 Downregulate Hey1 Expression in Blood and Lymphatic Endothelial Cells Without Altering Human Cell Viability.

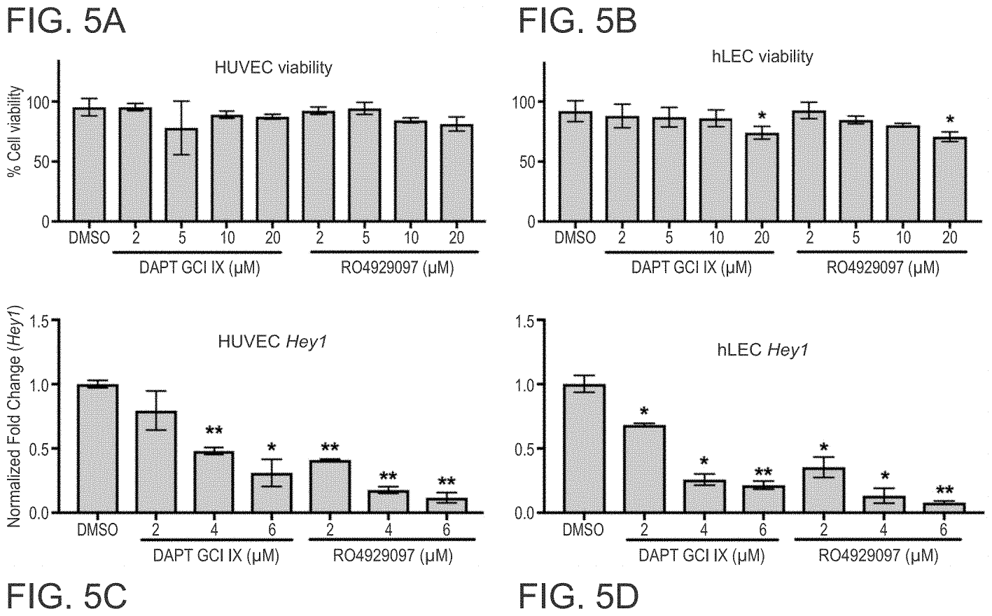

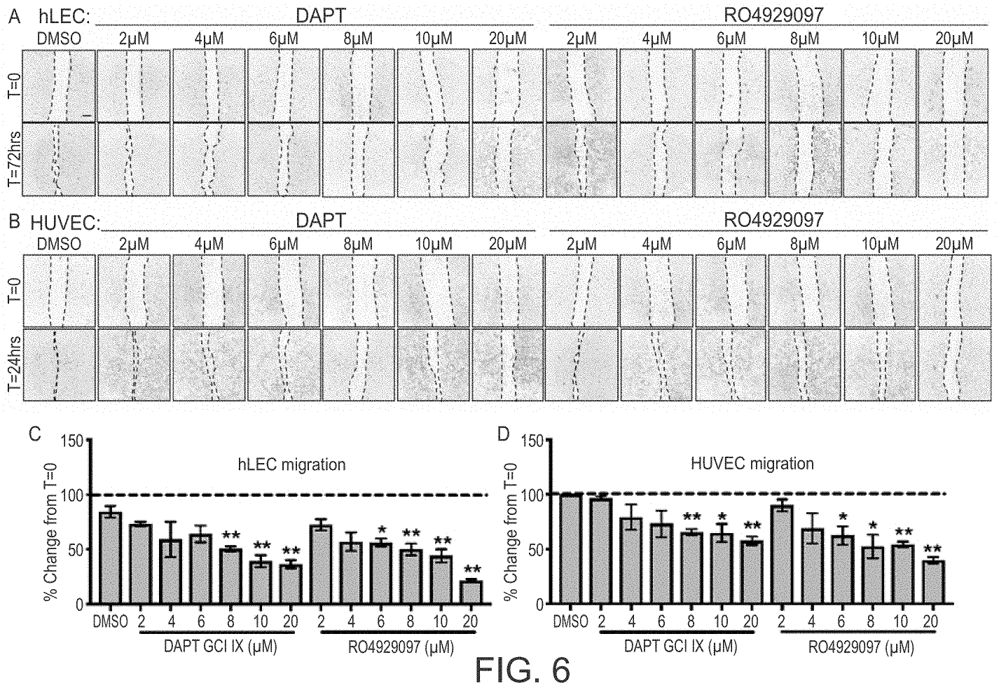

First, we confirmed that the GSIs in concentrations used in this study are successful in inhibiting the Notch signaling pathway in human blood (HUVECs) and lymphatic endothelial cells (hLECs) without altering their viability. Cell viability was not significantly different compared to untreated controls at concentrations less than 20 μM on either cell type. 20 μM of DAPT and RO4929097 reduced hLEC viability by almost 30% without significant changes observed at lower concentrations (FIG. 5A). Neither drug altered HUVEC cell viability at any of the concentrations tested (FIG. 5B). The expression of the NOTCH target gene Hey1 was significantly downregulated in both cell lines DAPT downregulated Hey1 expression in HUVECs beginning at 4 μM, while 2 μM of RO4929097 was sufficient to downregulate Hey1 (FIG. 5C). hLECs appeared more sensitive to GSIs, with both drugs causing inhibition at concentrations as low as 2 μM (FIG. 5D).

γ-Secretase Inhibitors Can Effectively Inhibit Angiogenesis of Both Human Blood and Lymphatic Cultured Endothelial Cells.

While the use of RAPT in the inhibition of migration and proliferation of blood endothelial cells has been well characterized¹⁶⁻¹⁹, to our knowledge there are no studies for the use of DAPT or RO4929097 on lymphatic endothelial cells. Therefore, we investigated the effectiveness of both these GSIs on the rate of migration of monolayer cultures of hLEC and HUVEC cells (FIG. 6A, 6B). 8 μM of DAPT reduced hLEC migration to about 50% and HUVEC migration to about 35% while RO4929097 was more effective at 6 μM in both cell types (FIG. 6C, 6D). RO4929097 reduced migration in hLECs by greater than 50% at lower concentrations compared to HUVECs.

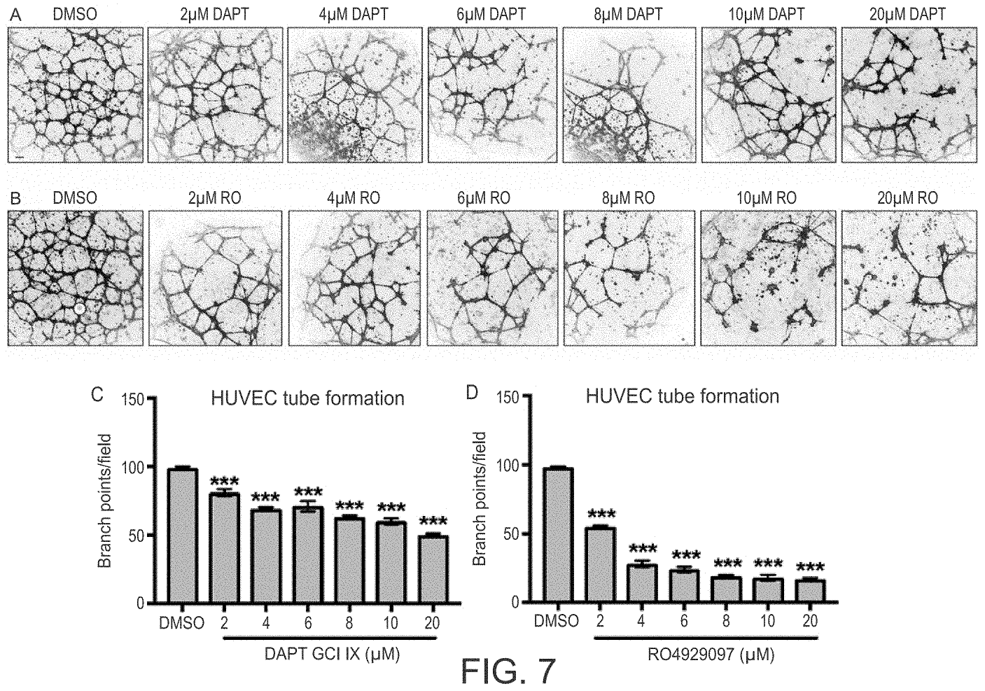

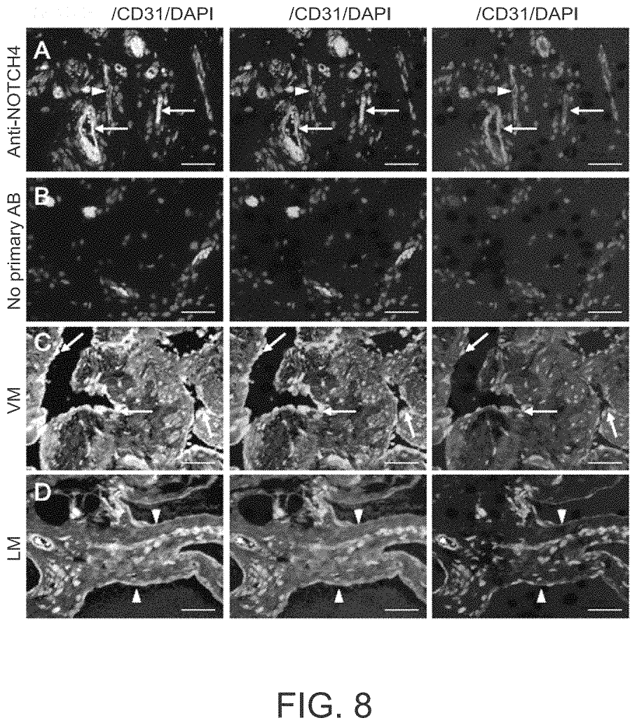

We determined the effect of GSIs on network formation of cultured human endothelial cells. Evaluation of tube formation by HUVEC in a matrigel matrix after treatment with increasing doses of DAPT and RO4909297 showed that network formation was dramatically reduced even with the lowest concentration (2 μM) of the GSIs (FIG. 7A, 6B). The number of branch points seen per field, an operational read-out of network formation assays, was significantly reduced with all concentrations of DAPT and RO4909297, with the latter being more potent in inhibiting network formation (FIG. 7C, 7D). Taken together, our results demonstrate that, HUVECs and hLECs are susceptible to both GSIs, and that lymphatic endothelial cells are more susceptible to GSIs compared to HUVECs.

Control antibody staining results are shown in Supplemental FIG. 1A-ID.

Discussion

A wide range of human extracranial vascular malformations involving lymphatic, venous and arterial vessels were examined and Notch proteins expression was demonstrated in each type. Although these results are qualitative, activated NOTCH1-ICD is more apparent in endothelial cells lining extracranial LM, VM and AVM compared to normal vessels within the same tissue. Full length NOTCH1 expression is observed in the endothelium of a majority of the LMs independent of whether the lesion was a focal (cystic LM) or multi-focal (generalized lymphatic anomaly/Gorham Stout). The broader expression of full length NOTCH1 than NOTCH1-ICD in the LM endothelium is consistent with the mechanism of NOTCH signaling, where Notch active cells are surrounded by ligand-expressing Notch inactive cells. NOTCH2 and NOTCH3 were expressed in endothelial and mural cells lining both VMs and LMs, and appeared to be increased compared to what we observed in normal vessels. NOTCH4-ICD was present in LM, but was sparsely expressed in both brain and extracranial AVM and VM. Our results suggest that Notch signaling plays a key role in human extracranial lymphatic, venous, and arteriovenous malformations and adds to the previously focused information on Notch overexpression in brain AVMs. We also found that, similar to those in the brain, extracranial AVMs showed interrupted αSMA and elastin layer surrounding malformed vessels.

The GSIs, DAFT and RO4909297, were effective in inhibiting angiogenesis in Notch-expressing HUVECs. RO4909297, which has been used in human clinical trials^{11, 14}, was more potent on a molar basis than DAPT, commonly used in vitro. Furthermore, hLECs were more susceptible to Notch inhibition than HUVECs. Our results are consistent with previous findings of GSI inhibition of HUVEC migration and tube formation²⁴⁻²⁶ and add to the information with comparative analysis between two GSIs across two different endothelial cells lines. It is reasonable to think that other GSIs would have similar effects. Although GSIs are known to be direct inhibitors of Notch, it also is possible that these drugs have effects downstream of Notch, e.g., on EphrinB2, which also is gamma secretase-processed when activated. Nonetheless, taken together our results present a strong impetus for targeting the Notch signaling pathway in the management and treatment of extracranial lymphatic, venous and arteriovenous malformations with GSIs. Combined loss of one allele of Notch1 and global deletion of Notch3 has been linked with retinal AVMs in mice²³. Notch2, while not described to be expressed in normal peripheral vascular cells, has a role in renal and eye capillary development²⁷. Notch-mediated communication between vascular endothelial cells and smooth muscle cells is critical during normal vascular development. Notch signaling is also important to the pathogenesis of vascular disease, and lies both upstream and downstream of critical vascular and lymphatic growth signals such as VEGF, EphB4/ephrinB2 and adrenomedullin/CLR signaling²⁸⁻³⁰. Data on the role of Notch in human vasculature are limited, but patients with CADASIL are known to have missense mutations in Notch3 or Jag1³¹. Patients with Alagille syndrome, a pleiotropic developmental disorder involving multiple organs, have demonstrated mutations in the Notch ligand Jag1²⁶. Our results support and extend several studies which have identified NOTCH1, 3 and 4 overexpression in AVMs of the central nervous system (CNS) in humans³³⁻³⁴. Possible tissue-, species-, and developmental stage-specific differences in the role of Notch signaling underline the need to study both inhibition and up-regulation in human vascular malformations.

Our observations are of particular interest, as previously noted, because several GSIs already have been through clinical trials, both in children and adults. Tolerated doses and schedules for oral administration have been well defined, particularly in older patients. Although lack of efficacy in Alzheimer's disease, the initial target population, led to shut-down of development of this class of drugs by the pharmaceutical industry, some efficacy was suggested in leukemias, solid tumors and in benign but locally aggressive desmoid tumors¹⁴⁻¹⁸. The maximum concentrations observed in pharmacokinetic studies of RO4929097 in patients with cancer were in the range of concentrations which we studied³⁵. Renewed interest in making drug available for one or more of these indications or for other rare disorders seems likely in the near future. In this disclosure, we studied only one of 5 GSIs which have been reported for use in human clinical trials. Preclinical testing of the other four drugs of this class may be of interest. Additional preclinical studies will be needed to detail the signaling pathways involved in development and progression of vascular malformations. However, we suggest that these can proceed concurrent with pilot phase II clinical trials of Notch inhibitors in patients with these disorders.

Materials and Methods

Patient Samples and IRB Approval:

All experimental protocols were carried out in accordance with relevant guidelines and regulations. All experimental protocols were approved by the IRB committees.

After obtaining an IRB waiver of approval, de-identified samples based on the database (VM [n=3], LM [n=2], extracranial AVM [n=6], brain AVM [n=3]) were obtained in paraffin blocks from UNC's Department of Surgical Pathology. An additional 8 LM samples were available from Columbia P&S. The extra-CNS biopsies came from variable locations including head and neck, chest, abdomen and extremities. An attempt was made to select the most recent specimens from patients who had not had prior sclerotherapy or malformation-directed medications. No patient had received GSIs for any disease. Because of the heterogeneity typical of histologic samples of vascular malformations, archival blocks were reviewed (SVS) to optimize sectioning of the part of the block most involved by the vascular malformation.

Cell Culture

Dermal human lymphatic endothelial cells (hLECs) from human neonates (HMVEC-dLyNeo-Der, Lonza CC-2812) and human umbilical venous endothelial cells (HUVEC, Lonza CC-2517) were used for in vitro assays (below) within 8 passages and maintained in EGM-2MV bullet kit (Lonza CC3202) and EGM bullet kit media respectively.

In Vitro Assays

Cell viability: To examine the effect of GSI on cell viability, hLEC and HUVEC cultures were incubated with effects of graded concentrations [2, 4, 6, 8, 10, 20 μM] of DAPT (Selleckchem, S2215) or RO4909297 (Selleckchem, S1575) [2-20 μM] for 24-72 hours. The percentage of viable cells was determined using the Countess automated cell counter (Thermofisher, C10227). Briefly, 80-90% confluent HUVEC and hLEC cultures were trypsinized, washed, and 10 μL of cell sample was mixed with an equal amount of 0.4% trypan blue stain and loaded onto the counting chamber.

Scratch migration assay—Migration assay was performed as per Liang et al., 2007³⁶, HUVECs and hLECs were grown to confluence in 24 well dishes, and then scratched with a 200 μL pipette tip. After scratching, the wells were rinsed with 1×PBS to remove non-adherent cells and then treated with control DMSO or increasing concentrations [2, 4, 6, 8, 10, 20 μM] of DAPT or RO4929097. Four fields per well were imaged at T=0 hrs and at T=24 hrs post-scratch or T=72 hrs for HUVECs and hLECs respectively using an Olympus IX-81 inverted microscope equipped with a QImaging Retiga 4000R camera at 4× magnification. The percent change in migration was calculated by measuring the open area of the scratch at the above mentioned time-points (ImageJ). Results shown are representative of four independent experiments with HUVECs and three for hLECs.

Tube formation assay—This assay was performed based on the protocol by Arnaoutova and Kleinman, 2010³⁷. Briefly, HUVEC cells were serum-starved after reaching 70-80% confluence, trypsinized, washed and plated at similar concentrations in a Growth Factor Reduced Matrigel Matrix (BD Biosciences 356230) coated 96-well plate and submerged in growth medium containing either control DMSO or increasing concentrations (2, 4, 6, 8, 10, 20 μM) of DAPT or RO4929097.

RNA and Quantitative RT-PCR

RNA was extracted from cultured HUVEC or hLEC cells either 24 hrs or 72 hrs post treatment respectively using TRIzol reagent (Ambion 15596026) followed by DNase (Promega M6101) treatment and cDNA prepared using iScript (BioRad 170-8890). Quantitative RT-PCR was done on StepOnePlus (ABI) using TaqMan Gene Expression Master Mix (ThermoFisher Scientific 4369016). Gene expression was assessed using human Single-tube assays (Thermo Fisher Scientific/Applied Biosystems): GAPDH (4310884E), ACTB (Hs99999903_m1) and HEY1 (Hs01114113_m1). Comparative ΔΔC_{T} method was used to analyze relative gene expression with ExpressionSuite Software (Thermo Fisher Scientific). Expression was normalized to housekeeping controls GAPDH and ACTB.

Immunofluorescence

Paraffin sections were stained as per Davis et al 2017²⁸. Briefly, they were rehydrated first followed by 20 minutes boiling for antigen retrieval in 10 mM Sodium citrate, 0.05% Tween 20, pH 6.0. They were then permeabilized in 1% triton X-100/PBS for 20 minutes and blocked in 5% NDS. Sections were incubated with primary antibodies, including mouse anti-smooth muscle alpha actin (1:200. Sigma-Aldrich A4700) and rabbit anti-activated Notch1 (1:100, Abeam ab8925) and Notch4 (1:100, Abcam ab33163) primary antibodies for 2 hours at room temperature. Sections were washed and incubated in secondary antibodies including donkey anti-rabbit Cy3 (Jackson ImmunoResearch 711-225-152), donkey anti-mouse Cyt (Jackson ImmunoResearch 715-545-151), and Bisbenzimide H 33258 Hoechst (Sigma-Aldrich B1155) at 1:250 at room temperature for 1 hour. The tissue sections were mounted in Prolong gold (Life Technologies P36934), Rabbit polyclonal antibodies against NOTCH2 (ab8926) and NOTCH3 (ab60087) were from Abcam (Cambridge, Mass.). Immunohistochemistry (IHC) carried in the Bond fully-automated slide staining system (Leica Biosystems Inc. Vista, Calif.). Slides were deparaffinized in Bond dewax solution (AR9222) and hydrated in Bond wash solution (AR9590). Antigen retrieval for all targets was performed at 1000C in Bond-epitope retrieval solution 1 pH 6.0 (AR9961) for 20 min. After pretreatment NOTCH2 (1:500) and NOTCH3 (1:200) were applied for 30 min. Detection was performed using Bond polymer refine detection system (DS9800). Stained slides were dehydrated and coverslipped. Positive and negative controls (no primary antibody) were included for each antibody. Elastin special stain was done at the UNC Animal Histopathology Core (AHC). Photomicrographs were reviewed (JB, KP, SVS) to confirm the types of cells (endothelium, muscle, pericyte) which were stained by each antibody. Notch staining was evaluated semi-quantitatively by comparing that of abnormal vessels within the malformation with positive and negative control tissues and with normal intra-lesional vessels. Abnormal vessels were categorized as showing more, less, or the same amount and intensity of staining than the controls. A rabbit polyclonal NOTCH4-ICD generated at Columbia P&S and goat polyclonal full-length NOTCH1 (1:100 R&D Systems AF1057) staining were previously described³⁸.

Image Acquisition

Images for the assays were taken on an Olympus microscope with cellSens software. Immunofluorescence images were acquired on a Nikon E800 fluorescence microscope with a Hammamatsu Orca CCD camera with Metamorph software (Molecular Devices Corp.). NOTCH2, 3, and elastin stained sections were digitally imaged (20× objective) in the Aperio ScanScope XT using line-scan camera technology (Leica Biosystems). Digital images were stored in the Aperio eSlide Manager software.

Statistical Analysis

All experiments were performed 3 or more times with 3 technical triplicates per assay and data are represented as a mean with SD or SEM. Significance was determined by Student t test (tail=2, type=2) with *P<0.05, **P<0.01 and ***P<0.001 considered significant.

### 7. REFERENCES

1. Francine Blei. Peripheral Vascular Anomalies, Malformations, and Vascular Tumors Thoracic Key. (Elsevier, 2013).
2. Blatt, J., McLean, T. W., Castellino, S. M. & Burkhart, C. N. A review of contemporary options for medical management of hemangiomas, other vascular tumors, and vascular malformations. Pharmacol. Ther. 139, 327-33 (2013).
3. Adams, D. M. et al. Efficacy and Safety of Sirolimus in the Treatment of Complicated Vascular Anomalies. Pediatrics 137, e20153257-e20153257 (2016).
4. Gridley, T. Notch Signaling in the Vasculature in Current topics in developmental biology 92, 277-309 (2010).
5. Krebs, L. T., Starling, C., Chervonsky, A. V & Gridley, T. Notch1 activation in mice causes arteriovenous malformations phenocopied by ephrinB2 and EphB4 mutants. Genesis 48, 146-50 (2010).
6. Uyttendaele, H., Ho, J., Rossant, J. & Kitajewski, J. Vascular patterning defects associated with expression of activated Notch4 in embryonic endothelium. Proc. Natl. Acad. Sci. 98, 5643-5648 (2001).
7. Carlson, T. R. et al. Endothelial expression of constitutively active Notch4 elicits reversible arteriovenous malformations in adult mice. Proc. Natl. Acad. Sci. U.S.A. 102, 9884-9 (2005).
8. Kim, Y. H. et al. Artery and vein size is balanced by Notch and ephrin B2/EphB4 during angiogenesis. Development 135, 3755-3764 (2008).
9. Murphy, P. A., Lu, G., Shiah, S., Bollen, A. W. & Wang, R. A. Endothelial Notch signaling is upregulated in human brain arteriovenous malformations and a mouse model of the disease. Lab. Invest. 89, 971-82 (2009).
10. ZhuGe, Q. et al. Notch-1 signalling is activated in brain arteriovenous malformations in humans. Brain 132, 3231-3241 (2009).
11. Fatima, A. et al. Murine Notch1 is required for lymphatic vascular morphogenesis during development. Dev. Dyn. 243, 957-964 (2014).
12. Bernier-Latmani, J. et al. DLL4 promotes continuous adult intestinal lacteal regeneration and dietary fat transport. J. Clin. Invest. 125, 4572-4586 (2015).
13. Shawber, C. J. & Kitajewski, J. Notch function in the vasculature: insights from zebrafish, mouse and man. Bioessays 26, 225-34 (2004).
14. Conic, V. et al. Safety and tolerability of the γ-secretase inhibitor avagacestat in a phase 2 study of mild to moderate Alzheimer disease. Arch. Neurol. 69, 1430-40 (2012).
15. Conic, V. et al. Targeting Prodromal Alzheimer Disease With Avagacestat: A Randomized Clinical Trial. JAMA Neurol. 72, 1324-33 (2015).
16. Fouladi, M. et al. Phase I Trial of MK-0752 in Children With Refractory CNS Malignancies: A Pediatric Brain Tumor Consortium Study. J. Clin. Oncol. 29, 3529-3534 (2011).
17. Lee, S. M. et al. Phase 2 study of RO4929097, a gamma-secretase inhibitor, in metastatic melanoma: SWOG 0933. Cancer 121, 432-440 (2015).
18. Villalobos, V. M. et al. Long-Term Follow-Up of Desmoid Fibromatosis Treated with PF-03084014, an Oral Gamma Secretase Inhibitor. Ann. Surg. Oncol. (2017). doi: 10.1245/s10434-017-6082-1
19. Papagiannaki, C. et al. Development of an angiogenesis animal model featuring brain arteriovenous malformation histological characteristics. J. Neurointerv. Surg. 9, 204-210 (2017).
20. Guo, Y. et al. Human brain arteriovenous malformations are associated with interruptions in elastic fibers and changes in collagen content. Turk. Neurosurg. 23, 10-5 (2012).
21. Hill-Felberg S., Wu, H. H., Toms, S. A. & Dehdashti, A. R. Notch receptor expression in human brain arteriovenous malformations. J. Cell. Mol. Med. 19, 1986-93 (2015).
22. Cai, X. et al. Mesenchymal status of lymphatic endothelial cell: enlightening treatment of lymphatic malformation. Int. J. Clin. Exp. Med. 8, 12239-51 (2015).
23. Kofler, N. M., Cuervo, H., Uh, M. K., Murtomäki, A. & Kitajewski, J. Combined deficiency of Notch1 and Notch3 causes pericyte dysfunction, models CADASIL, and results in arteriovenous malformations. Sci. Rep. 5, 16449 (2015).
24. Chigurupati, S. et al. Involvement of Notch Signaling in Wound Healing. PLoS One 2, e1167 (2007).
25. Wang, W.-M. et al. Epidermal Growth Factor Receptor Inhibition Reduces Angiogenesis via Hypoxia-Inducible Factor-1α and Notch1 in Head Neck Squamous Cell Carcinoma. PLoS One 10, e0119723 (2015).
26. Takeshita, K. et al. Critical role of endothelial Notch1 signaling in postnatal angiogenesis. Circ. Res. 100, 70-8 (2007).
27. McCright, B. et al. Defects in development of the kidney, heart and eye vasculature in mice homozygous for a hypomorphic Notch2 mutation. Development 128, 491-502 (2001).
28. Davis, R. B., Kechele, D. O., Blakeney, E. S., Pawlak, J. B. & Caron, K. M. Lymphatic deletion of calcitonin receptor-like receptor exacerbates intestinal inflammation. JCI Insight 2, e92465 (2017).
29. Silvestre, J.-S. & Mallat, Z. Arteries or Veins?: VEGF Helps EPCs Choose Their cAMP. Arterioscler. Thromb. Vasc. Biol. 26, 1934-1935 (2006).
30. Lanner, F. et al. Hypoxia-induced arterial differentiation requires adrenomedullin and notch signaling. Stem Cells Dev. 22, 1360-9 (2013).
31. Joutel, A. et al. Notch3 mutations in CADASIL, a hereditary adult-onset condition causing stroke and dementia. Nature 383, 707-710 (1996).
32. ZhuGe, Q. et al. Notch-1 signalling is activated in brain arteriovenous malformations in humans. Brain 132, 3231-41 (2009).
33. Hill-Felberg, S., Wu, H. H., Toms, S. A. & Dehdashti, A. R. Notch receptor expression in human brain arteriovenous malformations. J. Cell. Mol. Med. 19, 1986-1993 (2015).
34. Shoemaker, L. D. et al. Human brain arteriovenous malformations express lymphatic-associated genes. Ann. Clin. Transl. Neurol. 1, 982-995 (2014).
35. De Jesus-Acosta, A. et al. A phase II study of the gamma secretase inhibitor RO4929097 in patients with previously treated metastatic pancreatic adenocarcinoma. Invest. New Drugs 32, 739-45 (2014).
36. Liang, C.-C., Park, A. Y. & Guan, J.-L. In vitro scratch assay: a convenient and inexpensive method for analysis of cell migration in vitro. Nat. Protoc. 2, 329-333 (2007).
37. Arnaoutova, I. & Kleinman, H. K. In vitro angiogenesis: endothelial cell tube formation on gelled basement membrane extract. Nat. Protoc. 5, 628-635 (2010).
38. Vorontchikhina M A, et al. Unique patterns of Notch1, Notch4 and Jagged1 expression in ovarian vessels during folliculogenesis and corpus luteum formation. Gene Expr Patterns. 5, 701-709 (2005).

### 8. Generalized Statements of the Disclosure

The following numbered statements provide a general description of the disclosure and are not intended to limit the appended claims.

Statement 1: A method of treating vascular malformations in a subject which comprises administering to the subject a Notch inhibitor.

Statement 2: The method of Statement 1, wherein the vascular malformation is a venous malformation (VM), a lymphatic malformation (LM), a venolymphatic malformation (VLM) or an arteriovenous malformation (AVM).

Statement 3: The method of Statement 1 or 2, wherein the vascular malformation is an extracranial vascular malformation.

Statement 4: The method of Statement 1 or 2, wherein the vascular malformation is an intracranial vascular malformation.

Statement 5: The method of Statement 1, wherein the Notch inhibitor is a NOTCH 1, 2, 3 or 4 inhibitor.

Statement 6: The method of Statement 5, wherein the Notch inhibitor inhibits more than one Notch receptor protein.

Statement 7: The method of Statement 1, wherein the Notch inhibitor is a gamma secretase inhibitor (GSI).

Statement 8: The method of Statement wherein the Notch inhibitor is injected directly into a vascular malformation lesion.

Statement 9: The method of Statement 1, wherein the Notch inhibitor is delivered systemically.

Statement 10: The method of Statement 1, wherein the Notch inhibitor is delivered topically.

Statement 11: The method of Statement 1, wherein the Notch inhibitor is BMS-708163, BMS-906024, DAFT (GSI-IX), GM 136, GSI-953, LY3039478, LY450139, MK-0752, NIC5-15, PF-03084014, or R04929097 or a pharmaceutically acceptable salt thereof.

Statement 12: The method of Statement 1, wherein the subject is a child.

Statement 13: The method of Statement 1, wherein the subject is an adult.

Statement 14: A pharmaceutically acceptable formulation for the treatment of vascular malformations comprising a Notch inhibitor.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### Incorporation by Reference

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

1. A method of treating vascular malformations in a subject which comprises administering to the subject a Notch inhibitor.

2. The method of claim 1, wherein the vascular malformation is a venous malformation (VM), a lymphatic malformation (LM), a venolymphatic malformation (VLM) or an arteriovenous malformation (AVM).

3. The method of claim 1, wherein the vascular malformation is an extracranial vascular malformation.

4. The method of claim 1, wherein the vascular malformation is an intracranial vascular malformation.

5. The method of claim 1, wherein the Notch inhibitor is a NOTCH 1, 2, 3 or 4 inhibitor.

6. The method of claim 5, wherein the Notch inhibitor inhibits more than one Notch receptor protein.

7. The method of claim 1, wherein the Notch inhibitor is a gamma secretase inhibitor (GSI).

8. The method of claim 1, wherein the Notch inhibitor is injected directly into a vascular malformation lesion.

9. The method of claim 1, wherein the Notch inhibitor is delivered systemically.

10. The method of claim 1, wherein the Notch inhibitor is delivered topically.

11. The method of claim 1, wherein the Notch inhibitor is BMS-708163, BMS-906024, DAPT (GSI-IX), GSI 136, GSI-953, LY3039478, LY450139, MK-0752, NIC5-15, PF-03084014, or R04929097 or a pharmaceutically acceptable salt thereof.

12. The method of claim 1, wherein the subject is a child.

13. The method of claim 1, wherein the subject is an adult.

14. A pharmaceutically acceptable formulation for the treatment of vascular malformations comprising a Notch inhibitor.
